(19) United States
(12) Patent Application Publication
(10) Pub. No.: US 2024/0366100 A1
(43) Pub. Date: Nov. 7, 2024

(54) METHOD FOR EXTRACTING HEART RATE VARIABILITY FEATURE VALUE

(71) Applicant: VUNO Inc., Seoul (KR)

(72) Inventors: Youngjae SONG, Anyang-si (KR); Sungjae LEE, Seoul (KR)

(21) Appl. No.: 18/291,164

(22) PCT Filed: Aug. 10, 2022
(86) PCT No.: PCT/KR2022/011904

§ 371 Date: Jan. 22, 2024

(30) Foreign Application Priority Data
Sep. 24, 2021 (KR) 10-2021-0126071

(51) Int. Cl. A61B 5/024; A61B 5/00

(52) U.S. Cl. CPC A61B 5/02405; A61B 5/7267

(57) ABSTRACT

Disclosed is a method for extracting a heart rate variability (HRV) feature value performed by a computing device including one or more processors. The method includes acquiring first biosignal data measured during a first time period. The method includes outputting one or more heart rate variability feature values corresponding to a time period longer than the first time period by inputting the first biosignal data into a pre-trained neural network model.

### BACKGROUND

### Technical Field

The present disclosure relates to a method for measuring and analyzing heart-related biosignals, and more specifically, to a method for extracting a heart rate variability feature value using a neural network.

### Description of the Related Art

Generally, biosignals can be measured in various ways to determine a person's current state or predict his or her future state. For example, an electrocardiogram (ECG) signal or a photoplethysmography (PPG) signal can be measured to check conditions related to a person's heart.

The electrocardiogram signal is a record of electrical changes that occur locally due to heart activity. In addition, the photoplethysmography signal measures heart rate using a principle that the reflectance of light varies according to the expansion and contraction of blood vessels by irradiating light to body tissue.

Results of analysis of the electrocardiogram signal and the photoplethysmography signal can be used to diagnose various diseases. One of the information that can be acquired from these electrocardiogram signal and photoplethysmography signal is heart rate variability (HRV).

The heart rate variability refers to the degree of variation in heartbeat, and refers to the slight variation between one heart cycle and the next heart cycle. The heart rate variability is used to check the balance and activity of the autonomic nervous system, predict and evaluate the risk of developing stress-related diseases, evaluate resistance to disease, and confirm the effectiveness of treatment and follow-up tests.

Most of the feature values that can be derived from the heart rate variability have items which should be subjected to long-term measurement (for example, 5 minutes or more, 10 minutes or more, or 24 hours or more) to show reliable results.

Korean Patent Unexamined Publication No. 10-2018-0032829 discloses a heart rate signal measuring device.

### BRIEF SUMMARY

### Technical Problem

In order to extract heart rate variability feature values at a reliable level, a user must measure clean signals in a motionless position during a measurement period of heart-related biosignals such as electrocardiograms.

In this way, long-term measurement of the electrocardiogram signal or the photoplethysmography signal causes great inconvenience to the user, and there is a problem in that the quality of the signal may deteriorate due to various variables such as a user's movement.

Therefore, there is a need for research and development on methods for extracting reliable heart rate variability feature values using only short-term measurements.

The present disclosure is contrived in response to the above-mentioned background art, and has been made in an effort to provide a method for extracting a heart rate variability feature value using a neural network.

Technical objects of the present disclosure are not restricted to the technical object mentioned as above. Other unmentioned technical objects will be apparently appreciated by those skilled in the art by referencing the following description.

### Technical Solution

An exemplary embodiment of the present disclosure provides a method for extracting a heart rate variability (HRV) feature value performed by a computing device including one or more processors, which may include: acquiring first biosignal data measured during a first time period; and outputting one or more heart rate variability feature values corresponding to a time period longer than the first time period by inputting the first biosignal data into a pre-trained neural network model.

Alternatively, the pre-trained neural network model may be trained using a dataset generated based on a plurality of segments acquired by dividing second biosignal data measured during a second time period.

Alternatively, the outputting of the one or more heart rate variability feature values may include outputting the one or more heart rate variability feature values based on heart rate variability feature values corresponding to the plurality of segments, respectively, and a time period of each of the plurality of segments may be longer than the first time period.

Alternatively, the dataset may include a plurality of sub-segments acquired by dividing a first segment among the plurality of segments according to time as input data, and includes a heart rate variability feature value corresponding to third biosignal data extracted from the first segment as ground truth data of the input data.

Alternatively, the time period of each of the plurality of segments may correspond to the first time period.

Alternatively, when receiving an input indicating presence of an arrhythmia, the first time period in which the first biosignal data is measured may be set to be longer than a user who does not have the arrhythmia, or set as a time period up to a time point when a signal of a predefined pattern from the user is measured.

Alternatively, the inputting of the first biosignal data into the pre-trained neural network model, and outputting of the one or more heart rate variability feature values may include outputting the one or more heart rate variability feature values for each domain by inputting the first biosignal data into the pre-trained neural network model.

Alternatively, the domain may include at least one of a time domain, a frequency domain, and a nonlinear domain.

Alternatively, the pre-trained neural network model may include a plurality of sub-neural network models trained independently for each domain.

Another exemplary embodiment of the present disclosure provides a computer program stored in a computer-readable storage medium, wherein when the computer program is executed by one or more processors, the computer program allows the processor to perform a method for extracting a heart rate variability feature value, and the method may include: acquiring first biosignal data measured during a first time period; and outputting one or more heart rate variability feature values corresponding to a time period longer than the first time period by inputting the first biosignal data into a pre-trained neural network model.

Yet another exemplary embodiment of the present disclosure provides a computing device for extracting a heart rate variability feature value, which includes: a processor comprising one or more cores; and a memory including program codes executable in the processor, and the processor may be configured to acquire first biosignal data measured during a first time period, and output one or more heart rate variability feature values corresponding to a time period longer than the first time period by inputting the first biosignal data into a pre-trained neural network model.

### Advantageous Effects

According to an exemplary embodiment of the present disclosure, a heart rate variability feature value can be extracted using a neural network.

Effects which can be acquired in the present disclosure are not limited to the aforementioned effects and other unmentioned effects will be clearly understood by those skilled in the art from the following description.

### DESCRIPTION OF THE SEVERAL VIEWS OF THE DRAWINGS

Various aspects are now described with reference to the drawings and like reference numerals are generally used to designate like elements. In the following exemplary embodiments, for the purpose of description, multiple specific detailed matters are presented to provide general understanding of one or more aspects. However, it will be apparent that the aspect(s) can be executed without the detailed matters.

FIG. 1 is a block diagram of a computing device for extracting a heart rate variability feature value according to some exemplary embodiments of the present disclosure.

FIG. 2 is a schematic view illustrating a network function according to some exemplary embodiments of the present disclosure.

FIG. 3 is a diagram for describing a process of extracting a heart rate variability feature value through a neural network model according to some exemplary embodiments of the present disclosure.

FIG. 4 is a flowchart for describing a method for extracting a heart rate variability feature value according to some exemplary embodiments of the present disclosure.

FIG. 5 is a flowchart for describing a method for extracting a heart rate variability feature value according to some another exemplary embodiments of the present disclosure.

FIG. 6 is a flowchart for describing a method for extracting a heart rate variability feature value according to some yet another exemplary embodiments of the present disclosure.

FIG. 7 is a flowchart for describing a process of configuring a dataset for training the neural network model in the method for extracting a heart rate variability feature value according to some yet another exemplary embodiments of the present disclosure.

FIG. 8 is a flowchart for describing a method for acquiring biosignal data according to some exemplary embodiments of the present disclosure.

FIG. 9 is a flowchart for describing a method for acquiring biosignal data according to some another exemplary embodiments of the present disclosure.

FIG. 10 illustrates a simple and general schematic view of an exemplary computing environment in which the exemplary embodiments of the present disclosure may be implemented.

### DETAILED DESCRIPTION

Various embodiments will now be described with reference to drawings. In the present specification, various descriptions are presented to provide appreciation of the present disclosure. However, it is apparent that the embodiments can be executed without the specific description.

“Component”, “module”, “system”, and the like which are terms used in the specification refer to a computer-related entity, hardware, firmware, software, and a combination of the software and the hardware, or execution of the software. For example, the component may be a processing process executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

Moreover, the term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

Further, it should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

In addition, the term “at least one of A or B” should be interpreted to mean “a case including only A”, “a case including only B”, and “a case in which A and B are combined”.

Those skilled in the art need to recognize that various illustrative logical blocks, configurations, modules, circuits, means, logic, and algorithm steps described in connection with the embodiments disclosed herein may be additionally implemented as electronic hardware, computer software, or combinations of both sides. To clearly illustrate the interchangeability of hardware and software, various illustrative components, blocks, constitutions, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented embodiments is provided so that those skilled in the art of the present disclosure use or implement the present disclosure. Various modifications to the embodiments will be apparent to those skilled in the art. Generic principles defined herein may be applied to other embodiments without departing from the scope of the present disclosure. Therefore, the present disclosure is not limited to the embodiments presented herein. The present disclosure should be analyzed within the widest range which is coherent with the principles and new features presented herein.

In the present disclosure, a network function and an artificial neural network and a neural network may be interchangeably used.

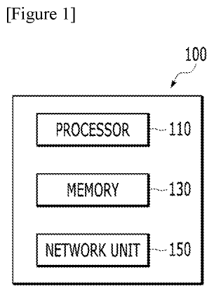

FIG. 1 is a block diagram of a computing device for extracting a heart rate variability (HRV) feature value according to some exemplary embodiments of the present disclosure.

A configuration of the computing device 100 illustrated in FIG. 1 is only an example simplified and illustrated. In an exemplary embodiment of the present disclosure, the computing device 100 may include other components for performing a computing environment of the computing device 100, and only some of the disclosed components may constitute the computing device 100.

The computing device 100 may include a processor 110, a memory 130, and a network unit 150.

The processor 110 may be constituted by one or more cores, and include processors for data analysis and deep learning, such as a central processing unit (CPU), a general purpose graphics processing unit (GPGPU), a tensor processing unit (TPU), etc., of the computing device. The processor 110 may read a computer program stored in the memory 130 and process data for machine learning according to an exemplary embodiment of the present disclosure. According to an exemplary embodiment of the present disclosure, the processor 110 may perform an operation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, the GPGPU, and the TPU of the processor 110 may process learning of the network function. For example, the CPU and the GPGPU may process the learning of the network function and data classification using the network function jointly. In addition, in an exemplary embodiment of the present disclosure, the learning of the network function and the data classification using the network function may be processed by using processors of a plurality of computing devices together. In addition, the computer program performed by the computing device according to an exemplary embodiment of the present disclosure may be a CPU, GPGPU, or TPU executable program.

According to an exemplary embodiment of the present disclosure, the memory 130 may store any type of information generated or determined by the processor 110 and any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

In respect to the network unit 150 according to an exemplary embodiment of the present disclosure, an arbitrary wired/wireless communication network that may transmit/receive arbitrary type data and signals may be included in the network expressed in the present disclosure.

The techniques described in this specification may also be used in other networks in addition to the aforementioned networks.

The processor 110 may acquire first biosignal data measured during a first time period to extract a heart rate variability feature value. Here, the first time period refers to a time period of a specific length, and may, for example, mean a short period (for example, less than 2 minutes and 30 seconds). For example, the first time period may be a time required to acquire first biosignal data including a specific signal from a user. For example, the first time period may mean the time period required for electrocardiogram measurement. As another example, the first time period may mean a time period required for photoplethysmography. As another example, the first time period may mean a time period required by input data used in an inference process of the trained neural network model.

Short term and long term in the present disclosure are terms used to express relatively long and short time periods. As an example, the short term may mean a time period of less than 2 minutes and 30 seconds, less than 1 minute, less than 30 seconds or less than 10 seconds, while the long term may mean a time period of 2 minutes and 30 seconds or more, 5 minutes or more, 10 minutes or more, 1 hour or more, or 24 hours or more. The short term may mean a relatively short time period compared to the long term.

Biosignal data is a biological signal that may be acquired from the human body and may mean data in electrical or magnetic form. The first biosignal data may mean biosignal data related to the heart, and the biosignal data related to the heart may include, for example, electrocardiogram data or photoplethysmography data. The ECG data may be acquired by attaching at least one lead to the skin of a user (e.g., a test subject) and measuring the result for a certain period of time. The photoplethysmography data may be acquired by attaching a sensor module including a light source and a photodetector to a part of the body (for example, a finger) and measuring the result for a certain period of time. The electrocardiogram data and the photoplethysmography data may each include information on a graph showing the intensity of the heart rate signal according to time. Specifically, the information in the graph may include a shape of the ECG curve amplifying minute current flowing in the user's heart muscle, a distance between waveforms of the ECG curve, a height of the ECG curve, an angle of the ECG curve, etc.

By analyzing the biosignal data, heart rate variability (HRV) may be acquired. The heart rate variability refers to the degree of variation in heartbeat, and refers to the slight variation between one heart cycle and the next heart cycle. One or more heart rate variability feature values may be extracted through analysis of the heart rate variability. The heart rate variability feature value may mean a value quantified according to a predetermined standard in a time domain, a frequency domain, or a nonlinear domain. For example, the heart rate variability feature values may include mRR, SDRR, mHR, SDHR, RMSSD, NN50, pNN50, VLF, LF, HF, pVLF, pLF, pHF, prcVLF, prcLF, powHF, nLF, nHF, LF/HF, SD1, SD2, ApEn, SampEn, D₂, Alpha1, Alpha2, Lmean, Lmax, REC, DET, and/or ShanEn.

Parameters representing feature values of heart rate variability may be exemplified in [Table 1] below.

TABLE 1

| | Parameters | Description |
|---|---|---|
| Time | mRR | Mean of RR intervals |
| domain | SDRR | Standard deviation of RR intervals |
| | mHR | Mean heart rates |
| | SDHR | Standard deviation of instantaneous heart rate |
| | | value |
| | RMSSD | Standard deviation for difference between |
| | | continuous RR intervals |
| | NN50 | Number of continuous RR interval pairs |
| | | showing difference of 50 ms or more |
| | pNN50 | Value acquired by dividing NN50 by total |
| | | number of RR intervals |
| Frequency | VLF | Peak value in very low frequency range |
| domain | | (0 to 0.04 Hz) |
| | LF | Peak value in low frequency range |
| | | (0.04 to 0.15 Hz) |
| | HF | Peak value in high frequency range |
| | | (0.15 to 0.4 Hz) |
| | pVLF | Absolute powers in VLF band |
| | pLF | Absolute powers in LF band |
| | pHF | Absolute powers in HF band |
| | prcVLF | Relative powers in VLF band, VLF (ms²)/total |
| | | power (ms²) × 100% |
| | prcLF | Relative powers in LF band, LF (ms²)/total |
| | | power (ms²) × 100% |
| | powHF | Relative powers in HF band, HF (ms²)/total |
| | | power (ms²) × 100% |
| | nLF | Power in LF band in normalized unit, LF |
| | | (ms²)/(LF + HF) (ms²) |
| | nHF | Power in HF band in normalized unit, HF |
| | | (ms²)/(LF + HF) (ms²) |
| | LF/HF | Ratio between LF and HF band powers |
| Nonlinear | SD1 | Standard deviation of Poincaré plot (short-term |
| domain | | fluctuation) |
| | SD2 | Standard deviation of Poincaré plot (long-term |
| | | fluctuation) |
| | ApEn | Approximate entropy |
| | SapmpEn | Sample entropy |
| | D₂ | Correlation dimension |
| | Alpha1 | Short-term fluctuations in detrended fluctuation |
| | | analysis (DFA) |
| | Alpha2 | Long-term fluctuations in detrendedm |
| | | fluctuation analysis (DFA) |
| | Lmean | Mean diagonal line length in recurrence plot |
| | | (RP) |
| | Lmax | Maximum diagonal line length in recurrence |
| | | plot (RP) |
| | REC | Recurrence rate: percentage of recurrence |
| | | points in RP |
| | DET | Determinism: percentage of recurrence points |
| | | forming diagonal line in RP |
| | ShanEn | Shannon entropy of probability distribution |
| | | of diagonal line length |

Although there are methods for measuring various types of biosignal data, hereinafter, for convenience of description, an electrocardiogram measurement method will be described as an example to describe the method for measuring the biosignal data.

The processor 110 may acquire first biosignal data measured from a separate measuring device, or directly acquire a first biosignal from at least one lead (not illustrated) included in the computing device 100. For example, the first biosignal data may be data used in the inference process of the neural network model 200.

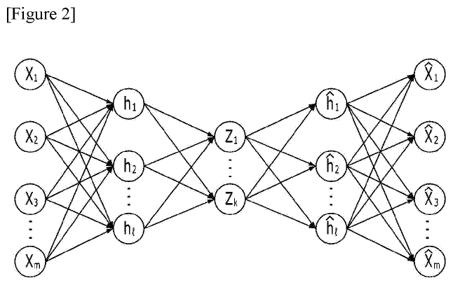

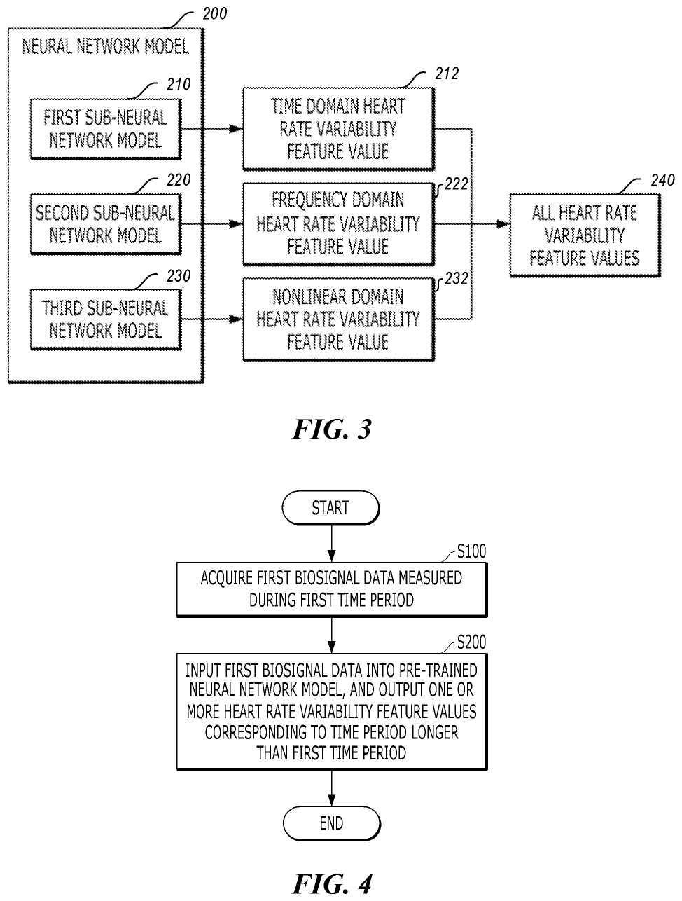

And, by inputting the first biosignal data into the pre-trained neural network model 200, one or more heart rate variability feature values may be output. Here, the neural network model 200 will be described later with reference to FIGS. 2 and 3.

FIG. 2 is a schematic view illustrating a network function according to an exemplary embodiment of the present disclosure.

Throughout the present specification, a computation model, a neural network, a neural network model, a sub-neural network model, a network function, and a neural network may be used as the same meaning. The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the links may relatively form a relationship of an input node and an output node. The concept of the input node is relative to the concept of the output node, and a predetermined node having an output node relationship with respect to one node may have an input node relationship in a relationship with another node, and a reverse relationship is also available. As described above, the relationship between the input node and the output node may be generated based on the link. One or more output nodes may be connected to one input node through a link, and a reverse case may also be valid.

In the relationship between an input node and an output node connected through one link, a value of the output node data may be determined based on data input to the input node. Herein, a link connecting the input node and the output node may have a weight. The weight is variable, and in order for the neural network to perform a desired function, the weight may be varied by a user or an algorithm. For example, when one or more input nodes are connected to one output node by links, respectively, a value of the output node may be determined based on values input to the input nodes connected to the output node and weights set in the link corresponding to each of the input nodes.

As described above, in the neural network, one or more nodes are connected with each other through one or more links to form a relationship of an input node and an output node in the neural network. A characteristic of the neural network may be determined according to the number of nodes and links in the neural network, a correlation between the nodes and the links, and a value of the weight assigned to each of the links. For example, when there are two neural networks in which the numbers of nodes and links are the same and the weight values between the links are different, the two neural networks may be recognized to be different from each other.

The neural network may consist of a set of one or more nodes. A subset of the nodes configuring the neural network may form a layer. Some of the nodes configuring the neural network may form one layer on the basis of distances from an initial input node. For example, a set of nodes having a distance of n from an initial input node may form n layers. The distance from the initial input node may be defined by the minimum number of links, which need to be passed to reach a corresponding node from the initial input node. However, the definition of the layer is arbitrary for the description, and a degree of the layer in the neural network may be defined by a different method from the foregoing method. For example, the layers of the nodes may be defined by a distance from a final output node.

The initial input node may mean one or more nodes to which data is directly input without passing through a link in a relationship with other nodes among the nodes in the neural network. Otherwise, the initial input node may mean nodes which do not have other input nodes connected through the links in a relationship between the nodes based on the link in the neural network. Similarly, the final output node may mean one or more nodes that do not have an output node in a relationship with other nodes among the nodes in the neural network. Further, the hidden node may mean nodes configuring the neural network, not the initial input node and the final output node.

In the neural network according to the embodiment of the present disclosure, the number of nodes of the input layer may be the same as the number of nodes of the output layer, and the neural network may be in the form that the number of nodes decreases and then increases again from the input layer to the hidden layer. Further, in the neural network according to another embodiment of the present disclosure, the number of nodes of the input layer may be smaller than the number of nodes of the output layer, and the neural network may be in the form that the number of nodes decreases from the input layer to the hidden layer. Further, in the neural network according to another embodiment of the present disclosure, the number of nodes of the input layer may be larger than the number of nodes of the output layer, and the neural network may be in the form that the number of nodes increases from the input layer to the hidden layer. The neural network according to another embodiment of the present disclosure may be the neural network in the form in which the foregoing neural networks are combined.

A deep neural network (DNN) may mean the neural network including a plurality of hidden layers, in addition to an input layer and an output layer. When the DNN is used, it is possible to recognize a latent structure of data. That is, it is possible to recognize latent structures of photos, texts, videos, voice, and music (for example, what objects are in the photos, what the content and emotions of the texts are, and what the content and emotions of the voice are). The DNN may include a convolutional neural network (CNN), a recurrent neural network (RNN), an auto encoder, Generative Adversarial Networks (GAN), a Long Short-Term Memory (LSTM), a transformer, a restricted Boltzmann machine (RBM), a deep belief network (DBN), a Q network, a U network, a Siamese network, a Generative Adversarial Network (GAN), and the like. The foregoing description of the deep neural network is merely illustrative, and the present disclosure is not limited thereto.

In the embodiment of the present disclosure, the network function may include an auto encoder. The auto encoder may be one type of artificial neural network for outputting output data similar to input data. The auto encoder may include at least one hidden layer, and the odd-numbered hidden layers may be disposed between the input/output layers. The number of nodes of each layer may decrease from the number of nodes of the input layer to an intermediate layer called a bottleneck layer (encoding), and then be expanded symmetrically with the decrease from the bottleneck layer to the output layer (symmetric with the input layer). The auto encoder may perform a nonlinear dimension reduction. The number of input layers and the number of output layers may correspond to the dimensions after preprocessing of the input data. In the auto encoder structure, the number of nodes of the hidden layer included in the encoder decreases as a distance from the input layer increases. When the number of nodes of the bottleneck layer (the layer having the smallest number of nodes located between the encoder and the decoder) is too small, the sufficient amount of information may not be transmitted, so that the number of nodes of the bottleneck layer may be maintained in a specific number or more (for example, a half or more of the number of nodes of the input layer and the like).

The neural network may be trained by at least one scheme of supervised learning, unsupervised learning, semi-supervised learning, and reinforcement learning. The training of the neural network may be a process of applying knowledge for the neural network to perform a specific operation to the neural network.

The neural network may be trained in a direction of minimizing an error of an output. In the training of the neural network, training data is repeatedly input to the neural network and an error of an output of the neural network for the training data and a target is calculated, and the error of the neural network is back-propagated in a direction from an output layer to an input layer of the neural network in order to decrease the error, and a weight of each node of the neural network is updated. In the case of the supervised learning, training data labelled with a correct answer (that is, labelled training data) is used, in each training data, and in the case of the unsupervised learning, a correct answer may not be labelled to each training data. That is, for example, the training data in the supervised learning for data classification may be data, in which category is labelled to each of the training data. The labelled training data is input to the neural network and the output (category) of the neural network is compared with the label of the training data to calculate an error. For another example, in the case of the unsupervised learning related to the data classification, training data that is the input is compared with an output of the neural network, so that an error may be calculated. The calculated error is back-propagated in a reverse direction (that is, the direction from the output layer to the input layer) in the neural network, and a connection weight of each of the nodes of the layers of the neural network may be updated according to the backpropagation. A change amount of the updated connection weight of each node may be determined according to a learning rate. The calculation of the neural network for the input data and the backpropagation of the error may configure a learning epoch. The learning rate is differently applicable according to the number of times of repetition of the learning epoch of the neural network. For example, at the initial stage of the learning of the neural network, a high learning rate is used to make the neural network rapidly secure performance of a predetermined level and improve efficiency, and at the latter stage of the learning, a low learning rate is used to improve accuracy.

In the training of the neural network, the training data may be generally a subset of actual data (that is, data to be processed by using the trained neural network), and thus an error for the training data is decreased, but there may exist a learning epoch, in which an error for the actual data is increased. Overfitting is a phenomenon, in which the neural network excessively learns training data, so that an error for actual data is increased. For example, a phenomenon, in which the neural network learning a cat while seeing a yellow cat cannot recognize cats, other than a yellow cat, as cats, is a sort of overfitting. Overfitting may act as a reason of increasing an error of a machine learning algorithm. In order to prevent overfitting, various optimizing methods may be used. In order to prevent overfitting, a method of increasing training data, a regularization method, a dropout method of inactivating a part of nodes of the network during the training process, a method using a bath normalization layer, and the like may be applied.

According to an embodiment of the present disclosure, a computer readable medium storing a data structure is disclosed.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

Throughout the present specification, a calculation model, a nerve network, the network function, and the neural network may be used with the same meaning. Hereinafter, the terms of the calculation model, the nerve network, the network function, and the neural network are unified and described with a neural network. The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes”. The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

FIG. 3 is a diagram for describing a process of extracting a heart rate variability feature value through a neural network model according to some exemplary embodiments of the present disclosure.

A configuration of the neural network model 200 illustrated in FIG. 3 is only an example shown through simplification. In some exemplary embodiments of the present disclosure, the neural network model 200 may include other components, and only some of the disclosed components may also constitute the neural network model 200. The heart rate variability feature value may mean a value quantified according to a predetermined standard in a time domain, a frequency domain, or a nonlinear domain. For example, the heart rate variability feature values may include mRR, SDRR, mHR, SDHR, RMSSD, NN50, pNN50, VLF, LF, HF, pVLF, pLF, pHF, prcVLF, prcLF, powHF, nLF, nHF, LF/HF, SD1, SD2, ApEn, SampEn, D₂, Alpha1, Alpha2, Lmean, Lmax, REC, DET, ShanEn, etc.

Referring to FIG. 3, the neural network model 200 may include a plurality of sub-neural network models independently trained for each domain. For example, the plurality of sub-neural network models may include a first sub-neural network model 210, a second sub-neural network model 220, and a third sub-neural network model 230. For example, the sub-neural network models 210, 220, and 230 may constitute one neural network model 200. At least one of a network structure, a training method, an input data format, or an output data format in the sub-neural network models 210, 220, and 230 may be the same. The sub-neural network models 210, 220, and 230 may share at least one of the network structure, the training method, the input data format, or the output data format. The neural network model 200 may generate a final output by ensembling outputs of the sub-neural network models 210, 220, and 230 or through an additional post-processing process on the outputs.

As another example, the sub-neural network models 210, 220, and 230 may exist independently of the neural network model 200.

In an exemplary embodiment of the present disclosure, each of the sub-neural network models 210, 220, and 230 may be trained based on heart rate variability feature values corresponding to different domains among the heart rate variability feature values.

The first sub-neural network model 210 may be a model trained to output one or more heart rate variability feature values corresponding to the time domain (at 212). Accordingly, when first biosignal data is input to the neural network model 200 by the computing device 100, the first sub-neural network model 210 may output one or more heart rate variability feature values corresponding to the time domain (at 212). For example, one or more heart rate variability feature values corresponding to the time domain may include mRR, SDRR, mHR, SDHR, RMSSD, NN50, pNN50, etc.

The second sub-neural network model 220 may be a model trained to output one or more heart rate variability feature values corresponding to the frequency domain (at 222). Accordingly, when first biosignal data is input to the neural network model 200 by the computing device 100, the second sub-neural network model 220 may output one or more heart rate variability feature values corresponding to the frequency domain (at 222). For example, one or more heart rate variability feature values corresponding to the frequency domain may include VLF, LF, HF, pVLF, pLF, pHF, prcVLF, prcLF, powHF, nLF, nHF, LF/HF, etc.

The third sub-neural network model 230 may be a model trained to output one or more heart rate variability feature values corresponding to the nonlinear domain (at 232). Accordingly, when first biosignal data is input to the neural network model 200 by the computing device 100, the third sub-neural network model 230 may output one or more heart rate variability feature values corresponding to the nonlinear domain (at 232). For example, one or more heart rate variability feature values corresponding to the nonlinear domain may include SD1, SD2, ApEn, SampEn, D₂, Alpha1, Alpha2, Lmean, Lmax, REC, DET, ShanEn, etc.

As in some exemplary embodiments described above in FIG. 3, the neural network model 200 may include a plurality of sub-neural network models and output one or more heart rate variability feature values for each domain. Additionally, the neural network model 200 may also output all heart rate variability feature values based on one or more heart rate variability feature values output for each domain (at 240). Methods of outputting all heart rate variability feature values include various methods including, for example, a method of summing the heart rate variability feature values output for each domain, a method of applying a predetermined weight to each of the heart rate variability feature values output for each domain, and summing them up, a method of applying a predetermined algorithm to the heart rate variability feature values output for each domain and summing them up, or a method of inputting the heart rate variability feature values output for each domain into an artificial intelligence model and using the output as the summed value.

Therefore, the processor 110 of the computing device 100 may output one or more heart rate variability feature values for each domain or for all domains according to the user's requirements by using the neural network model 200.

The method for extracting the heart rate variability feature value using the neural network model 200 in the computing device 100 described above with reference to FIGS. 1 to 3 will be described later.

FIG. 4 is a flowchart for describing a method for extracting a heart rate variability feature value according to some exemplary embodiments of the present disclosure.

Referring to FIG. 4, the processor 110 of the computing device 100 may acquire first biosignal data measured during a first time period (S100).

Here, the first time period may mean a short period (e.g., less than 30 seconds, less than 1 minute, or less than 2 minutes and 30 seconds). The examples of the time periods in this specification are merely examples used for the purpose of convenience of explanation and understanding, and the example time periods of these time periods may be variable depending on the type of applied application and the types of acquired parameters (e.g., heart rate variability feature values).

The first biosignal data may include electrocardiogram data or photoplethysmography data.

Further, the processor 110 may acquire first biosignal data measured from a separate measuring device, or directly acquire a first biosignal from at least one lead (not illustrated) included in the computing device 100.

Here, the processor 110 may preprocess the first biosignal data to input the first biosignal data into the neural network model 200. Preprocessing refers to preprocessing of input data to be input into the neural network model 200. For example, the processor 110 may derive a peak value of an R wave from the first biosignal data. Here, the peak value of the R wave may be derived using various peak value detection algorithms of the R wave, such as wavelet transform, pan-Tompkins algorithm, and deep learning algorithm, and the peak value detection algorithm of the R wave is not limited thereto.

As another example, the processor 110 may transform the first biosignal data from time domain data into frequency domain data or time-frequency domain data using Fourier transform.

Accordingly, the processor 110 may input the preprocessed first biosignal data into the neural network model 200.

The processor 110 of the computing device 100 may input the first biosignal data into the pre-trained neural network model 200 and output one or more heart rate variability feature values corresponding to a time period longer than the first time period (S200). For example, a neural network inference operation that outputs heart rate variability feature values measured over a relatively long period using biosignal data measured over a short period may be implemented using the trained neural network model 200.

Here, the neural network model 200 may include a plurality of sub-neural network models independently trained for each domain. Accordingly, the processor 110 of the computing device 100 may input the first biosignal data into the pre-trained neural network model 200 and output one or more heart rate variability feature values for each domain. Further, the processor 110 of the computing device 100 may use the neural network model 200 to combine one or more heart rate variability feature values output for each domain into one value and output all heart rate variability feature values.

According to some exemplary embodiments of the present disclosure, the neural network model 200 may include a supervised-learned model using a dataset including input data and ground truth data of the input data. The supervised-learned model may include a model learned using a neural network structure. The supervised-learned model may mean a model learned by reducing errors in the learning process using the ground truth data of the input data. The supervised-learned model may be a model learned using, for example, a convolutional neural network or a recurrent neural network.

In addition, the neural network model 200 may include a model consisting of a convolutional neural network, a recurrent neural network, as well as an attention mechanism model, and/or a transformer, alone or in combination.

The neural network model 200 may include an unsupervised-learned model using only input data. The unsupervised-learned model may include a model learned using the neural network structure. The unsupervised-learned model may mean a model learned by calculating an error by comparing input data and output data, and updating a connection weight of each node in each layer by backpropagating the calculated error in a reverse direction. The unsupervised-learned model may be, for example, a model learned using an auto encoder.

As in some exemplary embodiments described above in FIG. 4, the processor 110 of the computing device 100 may input the biosignal data measured over the short period into the neural network model 200 and output the heart rate variability feature value corresponding to the biosignal data measured over the long period, i.e., a heart rate variability feature value with high reliability.

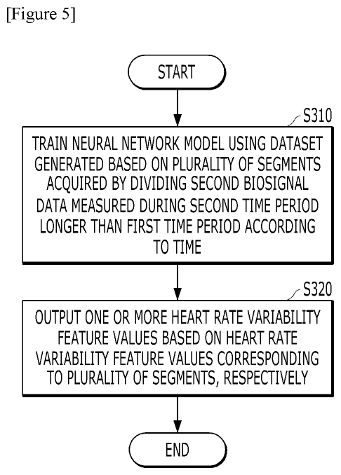

FIG. 5 is a flowchart for describing a method for extracting a heart rate variability feature value according to some another exemplary embodiments of the present disclosure.

Referring to FIG. 5, the processor 110 of the computing device 100 may acquire first biosignal data measured during a first time period.

The processor 110 of the computing device 100 may train the neural network model 200 using a dataset generated based on a plurality of segments acquired by dividing second biosignal data measured during a second time period longer than the first time period according to time (S310).

Here, the second time period may mean a long period (e.g., 5 minutes or more, 10 minutes or more, N hours or more, or 24 hours or more). Here, N corresponds to a natural number. The second time period may be a time required to secure data for training of the neural network model 200. For example, the second time period may mean a minimum time period required for training data used for training the neural network model 200. As another example, a sub-time period constituting the second time period may mean the minimum time period required for training data used for training the neural network model 200. For example, the second time period may mean a relatively long time period compared to the first time period. As another example, the second time period may mean a measurement time period of long-term measurement data, such as Holter data. As another example, the second time period may mean a time period in which a label in which a measurement result of the biosignal has a reliability value of a predetermined threshold level or more may be extracted. The second time period may have a variable value depending on which heart rate variability feature value is extracted. The second time period may be variable depending on at least one of the type of heart rate variability feature value, a biosignal acquisition method, a neural network model learning method, or the type of applied application. The second biosignal data may be data required for training the neural network model 200.

A segment in the present disclosure may mean a measurement time period in which a label with reliability of a predetermined threshold level or more may be extracted. A time period corresponding to the segment may also be variably determined depending on the type of heart rate variability feature value, the type of learning method, the type of applied application, etc., like other time periods.

In addition, according to some exemplary embodiments of the present disclosure, the plurality of segments may mean a time domain or unit of time in which the second biosignal data is continuously divided into a predetermined time period. For example, if the predetermined time period is 5 minutes, a first segment may be a domain of 0 to 5 (0 or more and less than 5) minutes in the second biosignal data, and a second segment may be a domain of 5 to 10 (5 or more and less than 10) minutes in the second biosignal data.

According to some other exemplary embodiments of the present disclosure, the plurality of segments may mean a time domain in which the second biosignal data is divided to be accumulated by a predetermined time period. For example, if the predetermined time period is 10 minutes, the first segment may be a domain of 0 to 10 (more than 0 and less than 10) minutes in the second biosignal data, and the second segment may be a domain of 0 to 20 (0 or more and less than 20) minutes in the second biosignal data.

In addition, according to some other exemplary embodiments of the present disclosure, the plurality of segments may mean a time domain or unit of time in which the second biosignal data is continuously divided at variable time periods. For example, the first segment may be a domain of 0 to 5 (0 or more and less than 5) minutes in the second biosignal data, and the second segment may be a domain of 5 to 15 (5 or more and less than 15) minutes in the second biosignal data.

In addition, according to some other exemplary embodiments of the present disclosure, the plurality of segments may mean a time domain or unit of time in which the second biosignal data is discontinuously divided into predetermined time periods. For example, if the predetermined time period is 5 minutes, the first segment may be a domain of 0 to 5 (0 or more and less than 5) minutes in the second biosignal data, and the second segment may be a domain of 10 to 15 (10 or more and less than 15) minutes in the second biosignal data.

Meanwhile, the processor 110 of the computing device 100 may acquire second biosignal data measured from a separate device, or directly acquire a second biosignal from at least one lead (not illustrated) included in the computing device 100. The computing device 100 may store second biosignal data in advance.

Therefore, the processor 110 of the computing device 100 divides the second biosignal data to construct a dataset, so that even when the amount of data measured over a long period of time is not sufficient, it is easy to construct the dataset, thereby training the neural network model regardless of the amount of data.

The processor 110 of the computing device 100 may output one or more heart rate variability feature values based on the heart rate variability feature values corresponding to the plurality of segments, respectively (S320).

Specifically, the processor 110 of the computing device 100 may input the first biosignal data into the pre-trained neural network model 200 and output one or more heart rate variability feature values based on the heart rate variability feature values corresponding to the plurality of segments, respectively.

In addition, the time period of each of the plurality of segments may be a third time period longer than the first time period which is a time required to acquire the first biosignal data including a specific signal from a user in the inference process of the neural network model 200. The third time period may be a time required to secure the data used for training of the neural network model 200. Specifically, the third time period may be a period between the first time period and the second time period which is the time required to secure the data for training of the neural network model 200. The third time period may mean a time period corresponding to the segment. For example, the third time period may mean a minimum time period required for the training data used for training the neural network model 200. As another example, a sub-time period constituting the third time period may mean a minimum time period required for the training data used for training the neural network model 200.

According to some exemplary embodiments of the present disclosure, the neural network model 200 may include a supervised-learned model using a dataset including input data and ground truth data of the input data. The supervised-learned model may include a model learned using a neural network structure. The supervised-learned model may mean a model learned by reducing errors in the learning process using the ground truth data of the input data. The supervised-learned model may be a model learned using, for example, a convolutional neural network or a recurrent neural network.

In addition, the neural network model 200 may include a model consisting of a convolutional neural network, a recurrent neural network, as well as an attention mechanism model, a transformer, etc., alone or in combination.

The neural network model 200 may include an unsupervised-learned model using only input data. The unsupervised-learned model may include a model learned using the neural network structure. The unsupervised-learned model may mean a model learned by calculating an error by comparing input data and output data, and updating a connection weight of each node in each layer by backpropagating the calculated error in a reverse direction. The unsupervised-learned model may be, for example, a model learned using an auto encoder.

As in some other exemplary embodiments described above in FIG. 5, the processor 110 of the computing device 100 trains the neural network model 200 using a plurality of segments with a longer time period than the first biosignal data to input the first biosignal data measured for a short period into the neural network model 200 and output one or more heart rate variability feature values having high reliability that may be acquired during long-term measurement. When performing inference on biosignal data measured over the short period through the learning method according to an exemplary embodiment of the present disclosure exemplarily illustrated in FIG. 5, a heart rate variability feature value with reliability corresponding to the case of measurement over a relatively long period may be output.

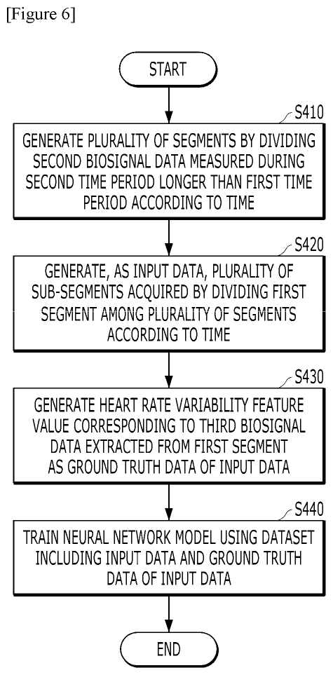

FIG. 6 is a flowchart for describing a method for extracting a heart rate variability feature value according to some yet another exemplary embodiments of the present disclosure.

Referring to FIG. 6, the processor 110 of the computing device 100 may acquire first biosignal data measured during a first time period.

And the processor 110 of the computing device 100 may divide the second biosignal data measured during a second time period longer than the first time period according to time to generate a plurality of segments (S410).

Here, the second time period may mean a long period (e.g., 10 minutes, or N hours or more). Here, N means a natural number.

In addition, according to some exemplary embodiments of the present disclosure, the plurality of segments may mean a time domain in which the second biosignal data is continuously or discontinuously divided into a predetermined time period. For example, if the predetermined time period is 5 minutes, a first segment may be a domain of 0 to 5 (0 or more and less than 5) minutes in the second biosignal data, and a second segment may be a domain of 5 to 10 (5 or more and less than 10) minutes in the second biosignal data. As another example, a plurality of segments may be discontinuously divided based on the value of the second biosignal data. In this case, in the example that the second time period is 10 minutes, if it is determined that the biosignal data from 5 minutes to 5 minutes and 30 seconds does not have a meaningful value due to arrhythmia, etc., the biosignal data from 0 to 5 minutes may be allocated to the first segment, and the biosignal data from 5 minutes 30 seconds to 10 minutes 30 seconds may be allocated to the second segment. In an exemplary embodiment of the present disclosure, the processor 110 may determine how to divide the acquired biosignal data by analyzing the values of the biosignal data.

According to some other exemplary embodiments of the present disclosure, the plurality of segments may mean a time domain in which the second biosignal data is divided to be accumulated by a predetermined time period. For example, if the predetermined time period is 10 minutes, a first segment may be a domain of 0 to 10 (0 or more and less than 10) minutes in the second biosignal data, and a second segment may be a domain of 0 to 20 (0 or more and less than 20) minutes in the second biosignal data.

The processor 110 of the computing device 100 may generate a plurality of sub-segments acquired by dividing the first segment among the plurality of segments according to time as input data (S420).

The time period of each of the plurality of sub-segments may correspond to the first time period. Accordingly, the processor 110 of the computing device 100 pre-trains the neural network model 200 using the first biosignal data and the input data corresponding to the time period to output one or more heart rate variability feature values having high accuracy.

The processor 110 of the computing device 100 may generate a heart rate variability feature value corresponding to the third biosignal data extracted from the first segment as ground truth data of the input data (S430).

Specifically, the processor 110 of the computing device 100 may extract the heart rate variability feature value using data acquired by preprocessing the third biosignal data extracted from the first segment. In addition, the processor 110 of the computing device 100 may generate the extracted the heart rate variability feature value of the third biosignal data as ground truth data of the input data.

The third biosignal data may be data measured during a sub-time period constituting the second time period for training the neural network model 200.

The processor 110 of the computing device 100 may train the neural network model 200 using a dataset including the input data and the ground truth data of the input data (S440).

Specifically, the processor 110 of the computing device 100 may generate each of the plurality of sub-segments acquired by dividing the first segment among the plurality of segments according to time as the input data, and the ground truth data of the input data for each of the plurality of sub-segments may be the heart rate variability feature value extracted using the data acquired by preprocessing the third biosignal data.

According to some exemplary embodiments of the present disclosure, the neural network model 200 may include a supervised-learned model using a dataset including input data and ground truth data of the input data. The supervised-learned model may include a model learned using a neural network structure. The supervised-learned model may mean a model learned by reducing errors in the learning process using the ground truth data of the input data. The supervised-learned model may be a model learned using, for example, a convolutional neural network or a recurrent neural network.

Specifically, the neural network model 200 may input each of the plurality of sub-segments as the input data into a neural network, and calculate an error between a value output through an output layer and the heart rate variability feature value corresponding to the third biosignal data which is the ground truth data of the input data, and may be pre-trained through supervised learning which updates a weight of each node of the neural network by backpropagating from the output layer to the input layer to reduce the error.

In addition, the neural network model 200 may include a model consisting of a convolutional neural network, a recurrent neural network, as well as an attention mechanism model, a transformer, etc., alone or in combination.

For example, the neural network model 200 may be a transformer including an encoder and a decoder. Specifically, the neural network model 200 may input each of the plurality of sub-segments as the input data into the encoder. Here, each of the plurality of sub-segments may be data in the form of an image.

In the neural network model 200, the encoder divides the input sub-segment into multiple sets, then divides each divided set into multiple sub-images, and processes the multiple divided sub-images in parallel to generate one set of output images. Then, the encoder generates a set of feature maps for each of one set of generated output images, merges each set of generated feature maps, and processes the merged feature maps to output an image vector. Here, various methods such as convolutional neural network, scale invariant feature transform (SIFT), histogram of oriented gradient (HOG), and speeded up robust features (SURF) may be used as a method for the encoder to process the sub-segment.

In the neural network model 200, the decoder may be repeatedly trained to apply the image vector output from the encoder to the attention mechanism to output the heart rate variability feature value corresponding to the third biosignal data, which is a target value.

In addition, the neural network model 200 may include a unsupervised-learned model using only input data. The unsupervised-learned model may include a model learned using the neural network structure. The unsupervised-learned model may mean a model learned by calculating an error by comparing input data and output data, and updating a connection weight of each node in each layer by backpropagating the calculated error in a reverse direction. The unsupervised-learned model may be, for example, a model learned using an auto encoder.

Meanwhile, the processor 110 of the computing device 100 may input the first biosignal data into the pre-trained neural network model 200 and output one or more heart rate variability feature values.

As in some yet another exemplary embodiments described above in FIG. 6, the processor 110 of the computing device 100 constitutes a dataset based on a plurality of segments acquired by dividing the second biosignal data measured during a second time period longer than the first time period according to time, and easily constitutes the dataset even when the amount of data measured for a long period of time is not sufficient to train the neural network model regardless of the amount of data.

When performing inference on biosignal data measured over the short period through the learning method according to an exemplary embodiment of the present disclosure exemplarily illustrated in FIG. 6, a heart rate variability feature value with reliability corresponding to the case of measurement over a relatively long period may be output.

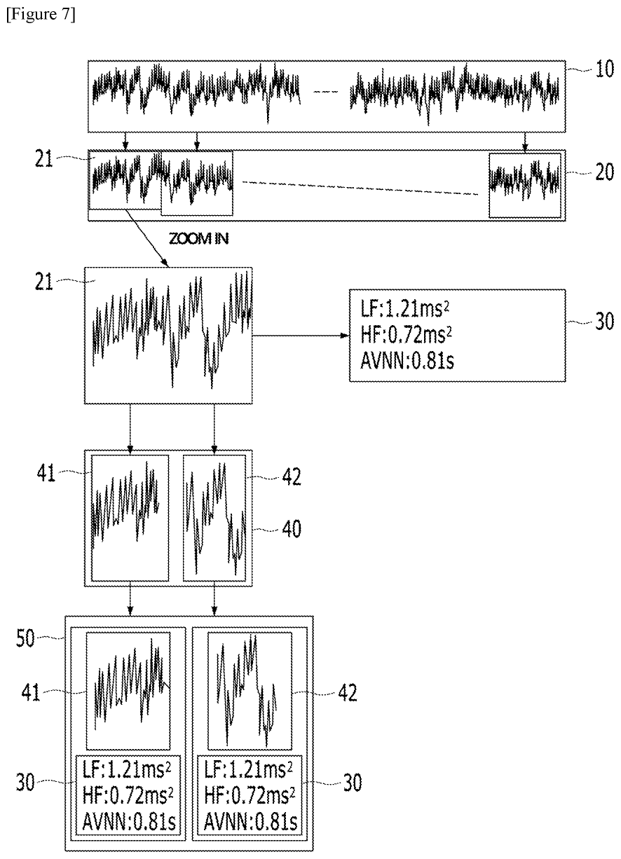

FIG. 7 is a flowchart for describing a process of configuring a dataset for training the neural network model in the method for extracting a heart rate variability feature value according to some yet another exemplary embodiments of the present disclosure.

Referring to FIG. 7, the processor 110 of the computing device 100 may divide second biosignal data 10 into a plurality of segments 20. Specifically, the processor 110 of the computing device 100 may divide the second biosignal data 10 into the plurality of segments 20 according to time.

Next, the processor 110 of the computing device 100 may extract third biosignal data from a first segment 21 of the plurality of segments 20. In addition, the processor 110 of the computing device 100 may extract a heart rate variability feature value 30 corresponding to the extracted third biosignal data.

Next, the processor 110 of the computing device 100 may divide the first segment 21 into a plurality of segments 40. Specifically, the processor 110 of the computing device 100 may divide the first segment 21 into the plurality of segments 40 according to time. Here, the plurality of sub-segments 40 may include a first sub-segment 41 and a second sub-segment 42. For example, the sub-segment may mean a sub-concept of the segment. As an example, the sub-segment may have a time period corresponding to 30 seconds or 1 minute or 2 minutes and 30 seconds. As an example, the sub-segment may have a time period corresponding to the time period of the input data used in the inference process.

Next, the processor 110 of the computing device 100 may generate a dataset 50 using a heart rate variability feature value 30 and the plurality of sub-segments 40. Specifically, the processor 110 of the computing device 100 may include the first sub-segment 41 and the second sub-segment 42 as the input data, and generate the dataset 50 including the heart rate variability feature value 30 corresponding to the extracted third biosignal data as ground truth data of the input data. As in some yet another exemplary embodiments described above in FIG. 7, the processor 110 of the computing device 100 constitutes the dataset based on the plurality of segments acquired by dividing the second biosignal data measured during the second time period which is a time required to secure the data for training the neural network model 200 to constitute multiple datasets, thereby training the neural network model to have high performance even when the amount of acquired data is small. The processor 110 of the computing device 100 may extract biosignal data from each of the plurality of segments 20 as well as the first segment 21 according to the process described above with reference to FIG. 7. In addition, the processor 110 of the computing device 100 may extract heart rate variability feature values corresponding to the biosignal data, respectively. Accordingly, the processor 110 of the computing device 100 may constitute the plurality of segments 20 as the dataset.

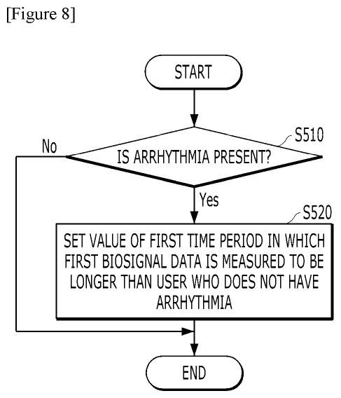

FIG. 8 is a flowchart for describing a method for acquiring biosignal data according to some exemplary embodiments of the present disclosure.

Referring to FIG. 8, the processor 110 of the computing device 100 may receive an input related to the presence or absence of an arrhythmia from the user before acquiring the first biosignal data (S510).

Specifically, the arrhythmia is a condition in which a heartbeat rhythm is irregular. Therefore, if the user has the arrhythmia, a situation may occur in which meaningful biosignal data cannot be acquired if the measurement time is not sufficient. Specifically, a situation may arise in which analysis is impossible due to acquisition of biosignal data without the user's heartbeat. In order to prevent such a situation, the processor 110 of the computing device 100 according to the present disclosure receives an input related to the presence or absence of the arrhythmia from the user to determine whether the first time period in which the first biosignal data is measured needs to be adjusted.

The processor 110 of the computing device 100 receives the input indicating that the arrhythmia is present (Yes in S510), the first time period (or the value of the first time period) in which the first biosignal data is measured may be set longer than for a user who do not have the arrhythmia (S520).

As in some exemplary embodiments described above in FIG. 8, the processor 110 of the computing device 100 adjusts the first time period (or the value of the first time period) in consideration of the presence of the arrhythmia disease to prevent the first biosignal data without the heartbeat from being measured.

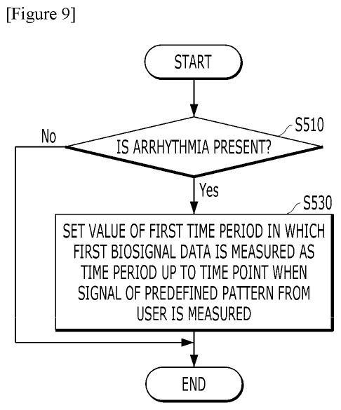

FIG. 9 is a flowchart for describing a method for acquiring biosignal data according to some another exemplary embodiments of the present disclosure.

Referring to FIG. 9, the processor 110 of the computing device 100 may receive an input related to the presence or absence of an arrhythmia from the user before acquiring the first biosignal data (S510).

Specifically, the arrhythmia is a condition in which a heartbeat rhythm is irregular. Therefore, if the user has the arrhythmia, a situation may occur in which meaningful biosignal data cannot be acquired if the measurement time is not sufficient. Specifically, a situation may arise in which analysis is impossible due to acquisition of biosignal data without the user's heartbeat. In order to prevent such a situation, the processor 110 of the computing device 100 according to the present disclosure receives an input related to the presence or absence of the arrhythmia from the user to determine whether the first time period in which the first biosignal data is measured needs to be adjusted.

The processor 110 of the computing device 100 receives the input indicating that the arrhythmia is present (Yes in S510), the first time period (or the value of the first time period) in which the first biosignal data is measured may be set as a time period up to a time point when a signal of a predefined pattern from the user is measured (S530).

Specifically, the processor 110 of the computing device 100 may set the signal of the predefined pattern as a signal that may derive a peak value of the R wave.

As in some exemplary embodiments described above in FIG. 9, the processor 110 of the computing device 100 adjusts the first time period (or the value of the first time period) in consideration of the presence of the arrhythmia disease to prevent the first biosignal data without the heartbeat from being measured.

It will also be apparent to those skilled in the art that as the steps or processes illustrated in FIGS. 4 to 9 are exemplary steps or processes and some of the steps or processes in FIGS. 4 to 9 may be omitted or there may be additional steps or processes are performed without departing from the scope of the present disclosure.

As in some exemplary embodiments described above in FIGS. 1 to 9, the processor 110 of the computing device 100 may input the biosignal data measured over the short period into the neural network model 200 and output the heart rate variability feature value corresponding to the biosignal data measured over the long period. That is, the processor 110 of the computing device 100 may output one or more heart rate variability feature values with high reliability that may be acquired during long-term measurement using biosignal data measured over a short period of time. Accordingly, since the processor 110 of the computing device 100 does not need to measure biosignals for a long time, biosignal measurement can be easily performed even with a wearable device, contributing to increased user convenience.

In addition, according to the existing technology, in particular, LF, HF, etc., among the heart rate variability feature values do not have reliable result values from electrocardiogram waveforms measured for a short period of time, such as less than 2 minutes and 30 seconds or less than 1 minute, but in the case of a heart rate variability feature value extraction scheme according to an exemplary embodiment of the present disclosure, there is an advantage of being able to extract sufficiently reliable heart rate variability feature values just by measuring the electrocardiogram over a relatively short period of time.

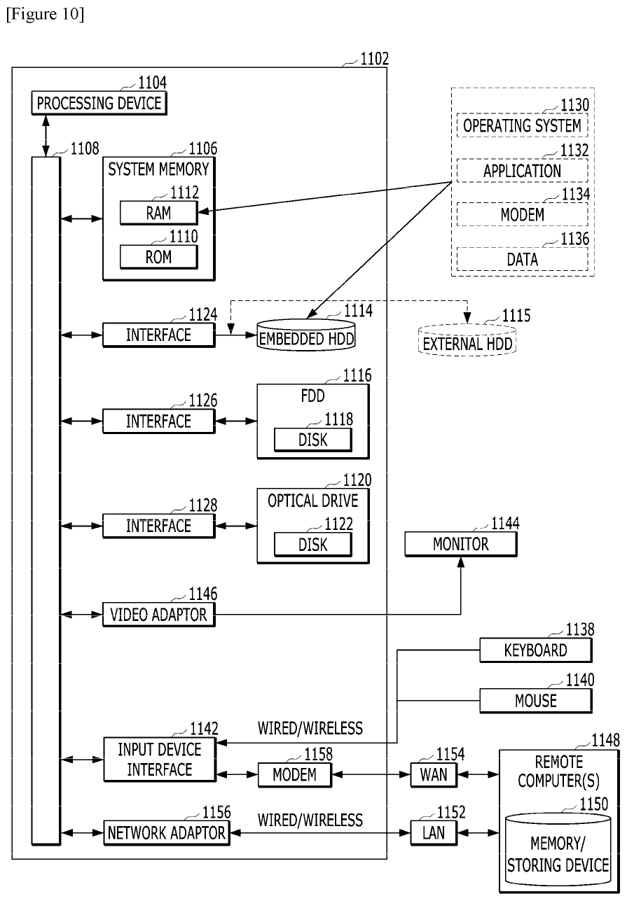

FIG. 10 is a simple and general schematic diagram illustrating an example of a computing environment in which the embodiments of the present disclosure are implementable.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An illustrative environment 1100 including a computer 1102 and implementing several aspects of the present disclosure is illustrated, and the computer 1102 includes a processing device 1104, a system memory 1106, and a system bus 1108. The system bus 1108 connects system components including the system memory 1106 (not limited) to the processing device 1104. The processing device 1104 may be a predetermined processor among various commonly used processors. A dual processor and other multi-processor architectures may also be used as the processing device 1104.

The system bus 1108 may be a predetermined one among several types of bus structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))—the embedded HDD 1114 being configured for exterior mounted usage within a proper chassis (not illustrated)—a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for extracting a heart rate variability (HRV) feature value performed by a computing device including one or more processors, the method comprising:
acquiring first biosignal data measured during a first time period; and
outputting one or more heart rate variability feature values corresponding to a time period longer than the first time period by inputting the first biosignal data into a pre-trained neural network model.

2. The method of claim 1, wherein the pre-trained neural network model is a model trained using a dataset generated based on a plurality of segments acquired by dividing second biosignal data measured during a second time period.

3. The method of claim 2, wherein the outputting of the one or more heart rate variability feature values includes:
outputting the one or more heart rate variability feature values based on heart rate variability feature values corresponding to the plurality of segments, respectively,
wherein a time period of each of the plurality of segments is longer than the first time period.

4. The method of claim 2, wherein the dataset includes a plurality of sub-segments acquired by dividing a first segment among the plurality of segments according to time as input data, and
wherein the dataset includes a heart rate variability feature value corresponding to a third biosignal data extracted from the first segment as ground truth data of the input data.

5. The method of claim 4, wherein the time period of each of the plurality of segments corresponds to the first time period.

6. The method of claim 1, wherein when an input indicating presence of an arrhythmia is received, the first time period in which the first biosignal data are measured is set to a longer time period than a user without arrhythmia, or to a time period up to a time point when a signal of a predefined pattern from the user is measured.

7. The method of claim 1, wherein the inputting of the first biosignal data into the pre-trained neural network model, and outputting of the one or more heart rate variability feature values includes:
outputting the one or more heart rate variability feature values for each domain by inputting the first biosignal data into the pre-trained neural network model.

8. The method of claim 7, wherein the domain includes at least one of a time domain, a frequency domain, and a nonlinear domain.

9. The method of claim 8, wherein the pre-trained neural network model includes a plurality of sub-neural network models trained independently for each domain.

10. A computer program stored in a computer-readable storage medium, the computer program causing one or more processors to perform a method for extracting a heart rate variability feature value when executed by the one or more processors, the method comprising:
acquiring first biosignal data measured during a first time period; and
outputting one or more heart rate variability feature values corresponding to a time period longer than the first time period by inputting the first biosignal data into a pre-trained neural network model.

11. A computing device for extracting a heart rate variability feature value, comprising:
a processor comprising one or more cores; and
a memory including program codes executable in the processor,
wherein the processor:
acquires first biosignal data measured during a first time period, and
outputs one or more heart rate variability feature values corresponding to a time period longer than the first time period by inputting the first biosignal data into a pre-trained
